(19) United States
(12) Patent Application Publication
(10) Pub. No.: US 2012/0204443 A1
(43) Pub. Date: Aug. 16, 2012

(54) Frequency Balance Footwear

(76) Inventor: Jon-Paul Nicholas Vertuccio, Lutz, FL (US)

(21) Appl. No.: 13/028,630

(22) Filed: Feb. 16, 2011

(51) Int. Cl. A43B 13/38; A61F 5/14

(52) U.S. Cl. 36/43; 36/140

(57) ABSTRACT

A footwear technology called Frequency Balance Footwear, is an footwear or insole that utilizes Mylar Holographic Disk Technology (small holograms). Each hologram disc is exposed to a Quantum Field Programming Generator which overlays or infuses specific blueprint frequencies for the human body which helps to promote physical posture, increased ability to achieve better balance, increased strength, and greater flexibility during any physical activity while wearing the Shoe and/or insoles.

### 1. CROSS-REFERENCE TO RELATED APPLICATIONS

Not Applicable (confirm)

### 2. STATEMENT REGARDING FEDERALLY SPONSORED RESEARCH OR DEVELOPMENT

Not Applicable (confirm)

### 3. BACKGROUND OF THE INVENTION

This new idea relates to any footwear that has Mylar holographic Disk technology embedded into or onto the shoes outer surface and/or insole of any shoe. Mylar Holographic Disks (the same substance used to keep static electricity from damaging, electrical components) has been imbedded with an electrical frequency that restores your body's electrical balance. This technology has the potential to increase an individual's ability to achieve better balance, increased strength, and greater flexibility by utilizing frequencies that occur in nature to improve the body's ability to absorb oxygen. Mylar holograms can be coded with the body's beneficial frequencies, which react with the body's energy. Each hologram disc is exposed to a Quantum Field Programming Generator which overlays or infuses specific blueprint frequencies for the human body.

Optimal Health and Peck performance occur when our body maintains ionic balance (the exchange between negative and positive charges) and free flowing pathways (harmony) at the optimum frequency. Eastern Medicine tapped this concept thousands of years ago, health practitioners developed healing treatments for the body and based it on its natural frequencies.

The Frequency Balance Footwear utilizes this technology in the shoe and insoles to maximize energy distribution and increases the body's ability to absorb and deliver more oxygen to various parts of the body as well as stabilize its balance.

### 4. BRIEF SUMMARY OF THE INVENTION

A purpose of the invention is to provide an individual the benefit of having the Mylar Hologram technology infused with frequencies embedded/made in to or on any shoe or insole to a shoe. This can help to promote balance, strength, and oxygen absorption during any physical activity all throughout the day.

One advantage is that the person only needs to have the Frequency Balance Footwear on during any physical activity which require shoes. Footwear is used everyday and this creates an extremely convenient way to combine shoes and insoles with Mylar holographic disk(s) that have been infused with the body's frequencies for all activities. Another advantage of the frequency balance footwear is when the Frequency Balance Footwear comes in contact with your body's energy field, it begins to resonate in accordance with each individual's biological energy system, creating a harmonic loop that optimizes your energy field, maintains maximum energy flow while it clears the pathways so the electro-chemical exchange functions like the well-tuned generator it was designed to be, resulting in immediate improved balance, increased core strength, greater flexibility, increased range of motion and overall well-being. With this technology now installed in shoes we have a better option of utilizing this technology without having any objects on the body, which may be beneficial for formal events or other athletic activities. Another benefit is to help with back posture when applied to any shoe of any kind.

The invention is any footwear that contains Mylar Holographic Disks Technology embedded in them to create an advantage in walking, standing, running, weightlifting, bicycling, baseball, football, golf, basketball, mountain climbing, and another sport(s) or activates.

The apparatus comprises (contains at least) 2-8 Mylar Holographic Disks (the same substance used to keep static electricity from damaging electrical components) Each hologram disc is exposed to a Quantum Field Programming Generator which overlays or infuses specific blueprint frequencies for the human body. This is technology is now combined on any single Shoe, or insole, needed to achieve optimum results so that the shoes are the focal point of ones balance and not any other object on the person(s) wearing them.

Further aspects of the invention will become apparent from consideration of the drawings and the ensuing description of preferred embodiments of the invention. A person skilled in the art will realize that other embodiments of the invention are possible and that the details of the invention can be modified in a number of respects, all without departing from the inventive concept. Thus, the following drawings and description are to be regarded as illustrative in nature and not restrictive.

### 5. BRIEF DESCRIPTION OF THE SEVERAL VIEWS OF THE DRAWINGS

The features of the invention will be better understood by reference to the accompanying drawings which illustrate presently preferred embodiments of the invention. In the drawings:

FIG. 1 is the side view of the Frequency Balance Footwear description, which consists of a shoe where Mylar holographic disks are embedded into the outer sole.

FIG. 2 Is an Arial view of my Frequency Balance Footwear insole that has Mylar holograms embedded into the insoles heals and or balls of the insole.

### 6. DETAILED DESCRIPTION OF THE INVENTION

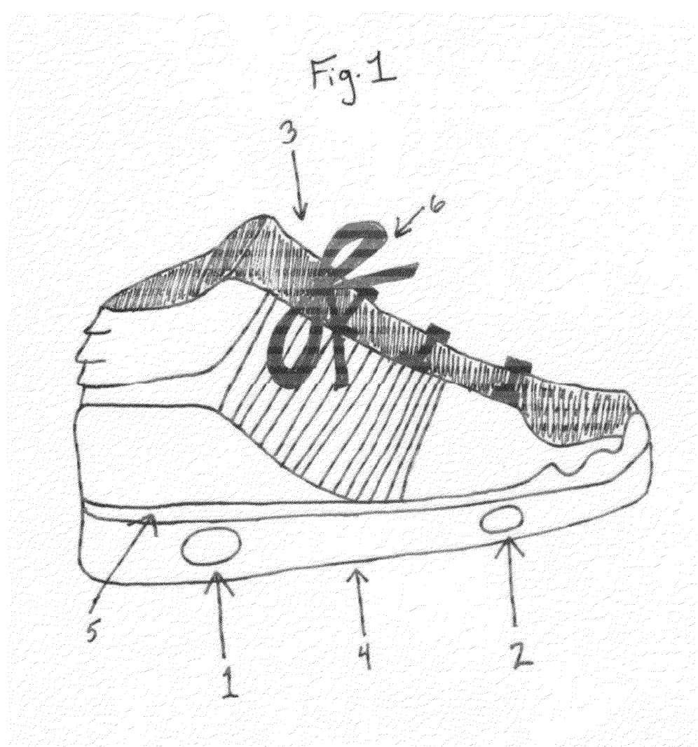

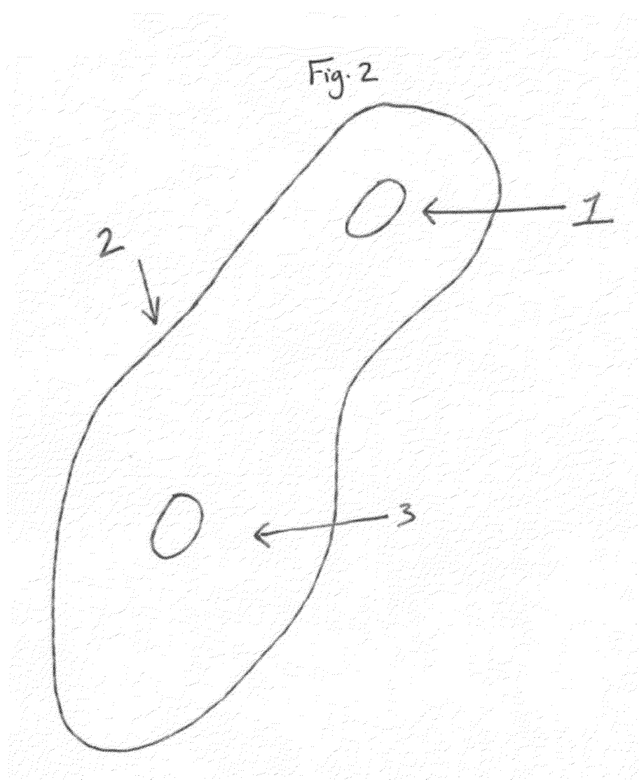

Referring to FIGS. 1-2, is giving a brief description of the shoe and insoles that have Mylar holograms. Each hologram disc is exposed to a Quantum Field Programming Generator which overlays or infuses specific blueprint frequencies for the human body.

Many variations of the invention will occur to those skilled in the art. Some variations include slippers, flip flops, tennis shoes, shoe insoles or any other form of footwear. All such variations are intended to be within the scope and spirit of the invention. The following reference numerals are used to indicate the parts and environment of the invention on the drawings of FIG. 1:

FIG. 1 comprises of sneaker made of all the major parts of a shoe. Items labeled 1 and 2 are the circular Mylar holograms infused with specific frequencies. Each hologram disc is exposed to a Quantum Field Programming Generator which overlays or infuses specific blueprint frequencies for the human body located on the Sole of the shoe labeled which has been labeled as item 4, which is sometimes referred to as the outer sole. Numbered item 5 is referred to as the midsole which is used to hold the outer sole to the rest of the shoe. Item numbered 6 are referred to as the shoe laces. Item numbered 3 are referred to as the tongue of the shoe. The Shoe is now completed in this view having the Mylar holograms located in the sole or “outersole” region as ladled in items 1 and 2.

Turing now to FIG. 2 as you can see is an insole to a shoe. The labeled portions of the insole identified as 1 is a circular Mylar Holograms that have been effused with frequencies. Each hologram disc is exposed to a Quantum Field Programming Generator which overlays or infuses specific blueprint frequencies for the human body and placed in the heal region of the insole. Item 3 is another circular Mylar hologram located in the balls of the foot region on the insole. Item 2 identifies the insole and outline of the insole used in footwear.

Although some embodiments are shown to include certain features, the applicant(s) specifically contemplate that any feature disclosed herein may be used together or in combination with any other feature on any embodiment of the invention. It is also contemplated that any feature may be specifically excluded from any embodiment of an invention.

What is claimed:

1. At least one shoe comprising of at least two Mylar holographic disk(s) that are infused with ion frequencies, applied as such to be in or on any shoes(S) heals, soles, tongues, and sides or any other location of the shoe, as to help stabilize the body's own natural frequencies.

2. Any insole for shoes in combination with at least two Mylar holographic disk(s) that are infused with ion frequencies, applied as such to be in or on any shoes insoles, heals, balls, and arches of the insole, as to help stabilize the body's own natural frequencies.

3. The shoe in claim 1 wherein the said Mylar holographic disk(s); is exposed to a Quantum Field Programming Generator which overlays or infuses specific blueprint frequencies for the human body.

4. The insole in claim 2 wherein the said Mylar holographic disk(s); is exposed to a Quantum Field Programming Generator which overlays or infuses specific blueprint frequencies for the human body.
